(19) United States
(12) Patent Application Publication
(10) Pub. No.: US 2023/0075007 A1
(43) Pub. Date: Mar. 9, 2023

(54) PHARMACEUTICAL FORMULATIONS OF CANNABIDIOL

(71) Applicant: IMPACT NATURALS, INC., San Diego, CA (US)

(72) Inventors: James LOWDER, San Diego, CA (US); Ramon SEVA, San Diego, CA (US); Vassili KOTLOV, San Diego, CA (US)

(21) Appl. No.: 17/818,310

(22) Filed: Aug. 8, 2022

Related U.S. Application Data
(60) Provisional application No. 63/231,190, filed on Aug. 9, 2021.
(60) Provisional application No. 63/367,122, filed on Jun. 27, 2022.

(51) Int. Cl. A61K 31/05; A61K 36/185; A61K 47/44; A61K 47/26

(52) U.S. Cl. CPC A61K 31/05; A61K 36/185; A61K 47/44; A61K 47/26

(57) ABSTRACT

The disclosure provides self-emulsifying compositions, comprising: (i) an active pharmaceutical ingredient (API) in an amount of 1-15% by weight of the composition; (ii) one or more non-polar solvents in an amount of 20-80% by weight of the composition; and (iii) one or more surfactants in an amount of 15-70% by weight of the composition, wherein the active pharmaceutical ingredient (API) comprises cannabidiol (CBD) and wherein the one or more non-polar solvents comprise a glyceride, together with methods of making and using the same.

### CROSS REFERENCE TO RELATED APPLICATIONS

This application claims the benefit of U.S. Provisional Application No. 63/367,122, filed Jun. 27, 2022, and U.S. Provisional Application No. 63/231,190, filed Aug. 9, 2021, the contents of which applications are incorporated herein by reference in their entirety.

### FIELD

The present invention relates to oral pharmaceutical formulations which contain cannabinoids, e.g., cannabidiol. More specifically, the invention relates to a self-emulsifying formulation suitable for encapsulation into a capsule dosage form, comprising one or more cannabinoids, e.g., cannabidiol, one or more non-polar solvents comprising glycerides, and surfactants. This formulation affords enhanced bioavailability of cannabidiol.

### BACKGROUND

Cannabidiol (CBD) is a naturally occurring cannabinoid in the Cannabis sativa plant. Cannabinoids are a class of diverse chemical compounds that act on cannabinoid receptors in cells that alter neurotransmitter release in the brain. There are at least 113 different cannabinoids isolated from Cannabis, exhibiting varied effects. Cannabidiol is the second most prevalent active ingredients of Cannabis extracts and has been studied for many different uses. It is known that cannabidiol lacks the psychoactive effects seen in many of the other cannabinoids including delta-9-tetrahydrocannabinol (THC). Cannabidiol may have potential as a treatment for a wide range of medical conditions including arthritis, diabetes, alcohol use disorders, multiple sclerosis, chronic pain, schizophrenia, post-traumatic stress disorder (PTSD), depression, rare white matter disorders, antibiotic-resistant infections, epilepsy, inflammation, and other neurological disorders. Cannabidiol is a component of pharmaceutical medications currently used to treat diseases such as multiple sclerosis and epilepsy. For example, an orally administered cannabidiol solution containing sesame oil (Epidiolex®) was approved by the US food and Drug Administration in 2018 as a treatment for two rare forms of childhood epilepsy: Lennox-Gastaut syndrome and Dravet syndrome. A nasal spray (Sativex®) containing both THC and cannabidiol is used for pain and muscle-tightness in people with multiple sclerosis.

Cannabidiol is a non-polar compound and has aqueous solubility of approximately 0.013 milligrams per milliliter. Furthermore, cannabidiol has low oral bioavailability and is inconsistently absorbed through the body's mucous membranes and from the digestive tract into the blood stream. Clinical studies have reported the oral bioavailability of cannabidiol as low as approximately 5-10% and absorption has been reported to fluctuate greatly depending on various factors such as the fat content of meals consumed by the study participants prior to dosing and other factors.

There is a need for improved formulations affording enhanced bioavailability of cannabidiol.

### BRIEF SUMMARY

It is surprisingly discovered that micro- or nano-emulsion formulations of cannabinoids, as described herein. are absorbed largely via the lymphatic system, allowing for enhanced bioavailability, enhanced distribution to the central nervous system, reduced first-pass effect and metabolism in the liver, reduced food effect, and reduced binding to plasma proteins, relative to formulations that are absorbed into the blood.

In one aspect, the present disclosure provides a self-emulsifying composition comprising:
(i) an active pharmaceutical ingredient (API) in an amount of 1-15% by weight of the composition;
(ii) one or more relatively non-polar solvents in an amount of 20-80% by weight of the composition; and
(iii) one or more surfactants in an amount of 15-70% by weight of the composition, wherein the active pharmaceutical ingredient (API) comprises cannabidiol (CBD) and wherein the one or more non-polar solvents comprise a glyceride. The active pharmaceutical ingredient (API) is free or substantially free of delta-9-tetrahydrocannabinol (THC). The composition self-emulsifies to form oil-in-water emulsions upon dilution with aqueous media, e.g., water or gastrointestinal fluids. In some embodiments, the one or more non-polar solvents comprises long-chain triglycerides, e.g., triglycerides comprising C16-22 fatty acids. In some embodiments, the one or more surfactants comprise polysorbate 80, polyoxyl 35, castor oil or a combination thereof.

In certain embodiments, the self-emulsifying composition comprises, by weight: a) about 10% API, b) about 22.5% sesame oil, c) about 22.5% of a mixture of glyceryl mono-, di-, and trilinoleate (or a mixture of corn oil mono-, di-, and triglycerides), c) about 22.5% polysorbate 80 and d) about 22.5% polyoxyl 35 castor oil.

In certain embodiments, the self-emulsifying composition comprises, by weight: a) about 10% API, b) about 27% PEG-6-glyceryl linoleate and/or corn oil PEG-6 esters, c) about 31.5% polysorbate 80 and d) about 31.5% polyoxyl 35 castor oil.

In certain embodiments, the self-emulsifying composition comprises, by weight, : a) about 10% API, b) about 36% sesame oil, c) about 18% of a mixture of glyceryl mono-, di-, and trilinoleate and/or a mixture of corn oil mono-, di-, and triglycerides, c) about 18% of a mixture of glyceryl mono and dicaprylocaprate, d) about 18% polysorbate 80 .

In another aspect, the disclosure provides a pharmaceutical composition comprising a self-emulsifying composition as described above. The pharmaceutical composition is an oral dosage form. The oral dosage form may be a capsule e.g., a soft gelatin capsule, a hard gelatin capsule, or a hydroxypropylmethyl cellulose (HPMC) capsule, a powder or a tablet. In some embodiments, the self-emulsifying composition is liquid and is filled into a capsule.

In another aspect, the disclosure provides a method of treating a condition selected from pain, anxiety, nausea, insomnia, and dysphoria comprising administering an effective amount of a pharmaceutical composition comprising a self-emulsifying composition as described above to a patient in need thereof.

In another aspect, the disclosure provides a method of relieving pain in a patient in need thereof, comprising administering an effective amount of a pharmaceutical composition comprising a self-emulsifying composition as described above in combination with opioid, wherein the coadministration of cannabidiol lowers the dose of opioid required to relieve pain.

In another aspect, the disclosure provides methods of enhancing the lymphatic absorption of a cannabinoid API, e.g., CBD or a mixture of CBD, CBG, and CBN, comprising administering a self-emulsifying composition as described herein.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DRAWINGS

FIG. 1A depicts levels of CBD in portal vein plasma for IN-07 (left) and IN-09 (right). FIG. 1B depicts levels of CBD in superior mesenteric lymph for IN-07 (left) and IN-09 (right). FIG. 1C depicts levels of CBD in portal vein plasma for a commercial formulation of CBD in a medium chain triglyceride vehicle (Medterra). FIG. 1D depicts levels of CBD in superior mesenteric lymph for a commercial formulation of CBD in a medium chain triglyceride vehicle (Medterra).

### DETAILED DESCRIPTION

Cannabidiol is water insoluble and has low oral bioavailability. The oral bioavailability of cannabidiol can be increased by self-emulsifying drug delivery systems (SEDDS) which are non-aqueous mixtures of non-polar solvents and surfactants. SEDDSs emulsify spontaneously to produce fine oil-in-water emulsions upon dilution with aqueous media, e.g., water or gastrointestinal fluids. The efficiency of oral delivery of cannabidiol from the SEDDS depends on many formulation-related parameters such as selection of non-polar solvents and surfactants, surfactant concentration, non-polar solvent/surfactant ratio. The size of the emulsion particles is also a crucial factor in self-emulsification performance because it determines the rate and extent of cannabidiol release as well as the stability of the emulsion.

In addition, there is another important consideration when formulating cannabinoids such as CBD into a self-emulsifying oral formulation. The oral bioavailability of CBD is affected by the first pass effect which is a phenomenon of drug metabolism whereby the concentration of a drug, specifically when administered orally, is greatly reduced before it reaches the systemic circulation. When CBD is taken orally, it is absorbed by the digestive system and enters the hepatic portal system. It is carried through the portal vein into the liver before it reaches the rest of the body. CBD undergoes extensive first pass metabolism and its metabolites are mostly excreted via the bile with a smaller proportion excreted through the kidneys.

The hepatic first-pass metabolism of CBD can be evaded to some extent by lymphatic transport pathway, which is a pathway through which lipids and lipophilic drugs are transported into systemic circulation. In the intestine, lipids are broken into their fatty acid and glycerol components and absorbed into the intestinal enterocyte, where they are reconstructed into triglycerides for packaging into chylomicrons. Chylomicrons are lipoprotein particles that consist of triglycerides, phospholipids, cholesterol, specific proteins and lipid soluble drugs if they are present. The chylomicron-associated lipids/drugs are then transported from the enterocyte into the lymphatic circulation, rather than the portal circulation, thus avoiding the first-pass metabolism in the liver. Shifting CBD absorption from the blood to the lymphatic system also facilitates transport of the drug to the brain, reduces food effects (i.e., variable absorption due to the contents of the GI tract), and potentially provides a better route of distribution and protection from metabolism than drug bound to albumin and other plasma proteins.

While the foregoing discussion focuses on CBD, other cannabinoids are subject to similar considerations and so can be advantageously administered in formulations according to the invention, e.g., to enhance bioavailability, reduce first-pass metabolism, and improve delivery to the central nervous system.

Self-emulsifying compositions as described herein are stable and form fine oil-in-water emulsions upon dilution with aqueous media, e.g., water or gastrointestinal fluids. It has been further found that the self-emulsifying compositions of the present invention promote the lymphatic transport of CBD. Without being bound to any theory, it is believed that the use of long-chain triglycerides, which require chylomicron formation to enter the body, in contrast to short and medium-chain triglycerides or no fats, provide lipid soluble drugs an alternative, preferable pathway to portal vein absorption.

The disclosure thus provides a self-emulsifying composition (Composition 1), comprising:
(i) an active pharmaceutical ingredient (API) in an amount of 1-15% by weight of the composition;
(ii) one or more non-polar solvents in an amount of 20-80% by weight of the composition; and
(iii) one or more surfactants in an amount of 15-70% by weight of the composition,
   wherein the active pharmaceutical ingredient (API) comprises a cannabinoid, e.g. cannabidiol (CBD), and wherein the one or more non-polar solvents comprise a glyceride.

For example, in particular embodiments the disclosure provides:
1.1. Composition 1, wherein the active pharmaceutical ingredient (API) is free or substantially free of delta-9-tetrahydrocannabinol (THC).
1.2. Composition 1 or 1.1 wherein the API contains less than 0.03%, less than 0.01%, less than 0.001%, less than 0.0001%, or less than 0.00001% THC by weight of delta-9-tetrahydrocannabinol (THC).
1.3. Any previous Composition, wherein the API is a CBD-rich hemp extract.
1.4.Any previous Composition, wherein the API comprises CBD.
1.5. Any previous composition wherein the API comprises CBG.
1.6. Any previous composition wherein the API comprises CBN.
1.7. Any previous Composition, wherein the API comprises CBD and CBG.
1.8. Any previous Composition, wherein the API comprises CBD and CBN.
1.9.Any previous Composition, wherein the API comprises CBD, CBG and CBN.
1.10. Any previous composition wherein the API comprises CBD and CBG in a weight ratio of 5-20:1 of CBD:CBG; e.g., about 10:1 CBD:CBG or about 25:2 CBD:CBG.
1.11. Any previous composition wherein the API comprises CBD and CBG in a weight ratio of 5-20:1 of CBD:CBN; e.g., about 10:1 CBD:CBN or about 27:2 CBD:CBN.
1.12. Any previous Composition wherein the API comprises CBD, CBG and CBN in a weight ratio of 1-10:1-10:1-10 (CBD:CBG:CBN).
1.13. Any previous Composition, wherein the cannabidiol present in the API is a synthetic cannabidiol.
1.14. Any previous Composition, wherein the API consists of cannibinoids selected from CBD, CBG, CBN, and mixtures thereof, e.g., wherein the API is substantially free of THC, e.g., wherein the API is substantially free of cannabinoids other than CBD, CBG, CBN, and mixtures thereof, e.g., wherein the API contains less than 0.1% of THC, e.g., wherein the API contains less than 0.1% of cannabinoids other than CBD, CBG, CBN, and mixtures thereof.
1.15. Any previous Composition, wherein the API consists CBD, CBG, and CBN, e.g., is substantially free of other cannabinoids, e.g., contains less than 0.1% of other cannabinoids.
1.16. Any previous Composition, wherein the API is present in an amount of 2-15%, 5-15%, 2-10%, 5-10%, 10-15%, 8-12%, or about 10% by weight of the composition.
1.17. Any previous Composition, wherein the composition emulsifies spontaneously to produce fine oil-in-water emulsions upon dilution with aqueous media, e.g., water or gastrointestinal fluids.
1.18. Any previous Composition, wherein the emulsion formed upon dilution with gastrointestinal fluids has an oil-phase particle-size distribution where an average oil-phase droplet diameter is less than 1000 nm, e.g., less than 500 nm, less than 100 nm or less than 50 nm.
1.19. Any previous Composition, wherein the one or more non-polar solvents are present in an amount of 30-70%, or 35-65% by weight of the composition.
1.20. Any previous Composition, wherein the glyceride is monoglyceride, diglyceride, triglyceride or mixtures thereof.
1.21. Any previous Composition, wherein the glyceride comprises triglyceride.
1.22. Any previous Composition, wherein the glyceride contains C₁₃ to C₂₄ fatty acids.
1.23. Any previous Composition, wherein the glyceride contains long-chain (C₁₆ to C₂₂) fatty acids.
1.24. Any previous Composition, wherein the glyceride contains C₁₆ and/or C₁₈ fatty acids.
1.25. Any previous Composition, wherein the glyceride is free of medium-chain (C₆to C₁₂) triglycerides.
1.26. Any previous Composition, wherein the glyceride contains saturated or unsaturated fatty acid.
1.27. Any previous Composition, wherein the one or more non-polar solvents comprise (i) sesame oil and (ii) glyceryl mono-, di-, and trilinoleate and/or a mixture of corn oil mono-, di-, and triglycerides.
1.28. Any previous Composition, wherein the one or more non-polar solvents comprise (i) sesame oil and (ii) glyceryl mono-, di-, and tri-oleate and linoleate and/or a mixture of mono-, di-, and triglycerides of corn oil fatty acids; or wherein the one or more non-polar solvents comprise (i) sesame oil and (ii) a mixture of corn oil mono-, di-, and triglycerides.
1.29. Any previous Composition, wherein the one or more non-polar solvents comprise (i) sesame oil, (ii) a mixture of glyceryl mono-, di-, and tri-oleate and linoleate and/or a mixture of mono-, di-, and triglycerides of corn oil fatty acids; and (iii) a mixture of glyceryl mono and dicaprylocaprate.
1.30. Any previous Composition, wherein the one or more non-polar solvents comprise PEG-6-glyceryl linoleate.
1.31. Any previous Composition, wherein the one or more surfactants are present in an amount of 20-65% by weight of the composition.
1.32. Any previous Composition, wherein the one or more surfactants have a HLB of about 8 to 16.
1.33. Any previous Composition, wherein the total surfactants in the composition have an overall HLB of about 12 or higher.
1.34. Any previous Composition, wherein the one or more surfactants are non-ionic surfactants.
1.35. Any previous Composition, wherein the one or more surfactants comprise polysorbate 80, polyoxyl 35 castor oil or a combination thereof.
1.36. Any previous Composition, wherein the one or more surfactants comprise polysorbate 80.
1.37. Any previous Composition, wherein the one or more surfactants comprise polysorbate 80 and polyoxyl 35 castor oil.
1.38. Any previous Composition, wherein the composition further comprises one or more non-polar antioxidants, optionally selected from mixed tocopherols, quercetin, curcumin or other suitable oil-soluble antioxidants.
1.39. Any previous Composition, wherein the composition further comprises anti-bacterial and/or anti-fungal agents, optionally wherein the anti-bacterial and/or anti-fungal agents are selected from thymol, carvacrol, lauric acid, or other naturally occurring anti-bacterial and/or anti-fungal agents.
1.40. Any previous Composition, wherein the self-emulsifying composition comprises, by weight: a) 8-12% API; b) 20-25% sesame oil; c) 20-25% of a mixture of glyceryl mono-, di-, and tri-oleate and linoleate (or a mixture of mono-, di-, and triglycerides of corn oil fatty acids); d) 20-25% polysorbate 80; and e) 20-25% polyoxyl 35 castor oil.
1.41. Any previous Composition, wherein the self-emulsifying composition comprises, by weight: a) 8-12% API; b) 25-30% mono-, di- and triglycerides and PEG-6 (MW 300) mono- and diesters of linoleic acid; c) 29-34% polysorbate 80; and d) 29-34% polyoxyl 35 castor oil.
1.42. Any previous Composition, wherein the self-emulsifying composition comprises, by weight: a) 8-12% API; b) 34-38% sesame oil; c) 16-20% of a mixture of glyceryl mono-, di-, and tri-oleate and linoleate and/or a mixture of mono-, di-, and triglycerides of corn oil fatty acids; d) 16-20% of a mixture of glyceryl mono and dicaprylocaprate, and e) 16-20% polysorbate 80.
1.43. Any previous Composition, wherein the self-emulsifying composition comprises, by weight: a) about 10% API, b) about 22.5% sesame oil, c) about
22.5% of a mixture of glyceryl mono-, di-, and tri-oleate and linoleate and/or a mixture of corn oil mono-, di-, and triglycerides; d) about 22.5% polysorbate 80, and e) about 22.5% polyoxyl 35 castor oil.
1.44. Any previous Composition, wherein the self-emulsifying composition comprises, by weight:
   5-15%, e.g., about 10% CBD;
   20-25%, e.g., about 22.5% sesame oil;
   20-25%, e.g., about 22.5% mono-, di- and triglycerides of fatty acids, e.g., fatty acids from corn oil, e.g., mainly linoleic (C_{18:2}) and oleic (C_{18:1}) acids, e.g., wherein the diester fraction is predominant (e.g., glyceryl monolinoleate USP-NF);
   20-25%, e.g., about 22.5% polysorbate 80;
   20-25%, e.g., about 22.5% polyoxyl-35 castor oil.
1.45. Any previous Composition, wherein the self-emulsifying composition comprises, by weight:
   5-15%, e.g., about 10% CBD;
   25-30%, e.g., about 27% mono-, di- and triglycerides and PEG-6 (MW 300) mono- and diesters of linoleic (C_{18:2}) acid (e.g., linoleoyl polyoxyl-6 glycerides USP-NF);
   28-35%, e.g., about 31.5% polysorbate 80;
   28-35%, e.g., about 31.5% polyoxyl-35 castor oil.
1.46. Any previous Composition wherein, after oral administration, at least 40%, e.g., at least 50%, e.g., at least 60%, of the API absorbed is absorbed via the lymphatic system rather than the portal vein absorption, e.g., as measured in rats with cannulated intestinal lymph duct and portal veins, e.g., as described in Example 2.
1.47. Any previous Composition wherein the liver metabolism of the API within one hour of oral administration is reduced relative to the liver metabolism of the API within one hour of oral administration of a formulation that is not a self-emulsifying composition.
1.48. Any previous Composition wherein, after oral administration, the total bioavailability of the API is at least 70%, e.g., at least 80%, e.g., at least 90%.
1.49. Any previous Composition wherein the bioavailability after oral administration is substantially the same, e.g., ±10%, in fed and fasted subjects.
1.50. Any previous Composition wherein, after oral administration, the API is present in the cerebrospinal fluid (CSF) at levels greater than the levels in the plasma.
1.51. Any previous Composition in a unit dose form for oral administration, e.g., in a capsule, e.g., a soft gelatin (softgel) capsule.
1.52. Any previous Composition in a unit dose form for oral administration, e.g., in a capsule, e.g., a soft gelatin (softgel) capsule, comprising API in the amount of 20-100 mg.
1.53. Any previous Composition in a unit dose form for oral administration, e.g., in a capsule, e.g., a soft gelatin (softgel) capsule, wherein the API comprises 10-50 mg CBD.
1.54. Any previous Composition in a unit dose form for oral administration, e.g., in a capsule, e.g., a soft gelatin (softgel) capsule, wherein the API comprises 10-50 mg CBD and 1-5 mg CBG.
1.55. Any previous Composition in a unit dose form for oral administration, e.g., in a capsule, e.g., a soft gelatin (softgel) capsule, wherein the API comprises 10-50 mg CBD and 1-5 mg CBN.
1.56. Any previous Composition in a unit dose form for oral administration, e.g., in a capsule, e.g., a soft gelatin (softgel) capsule, wherein the API comprises 10-50 mg CBD, 1-5 mg CBG, and 1-5 mg CBN.

In a further embodiment, the invention provides
a. a method of enhancing delivery of a cannabinoid, e.g., CBD, CBG, and/or CBN, to the lymphatic system;
b. a method of enhancing delivery of a cannabinoid, e.g., CBD, CBG, and/or CBN, to the central nervous system;
c. a method of reducing first pass metabolism of a cannabinoid, e.g., CBD, CBG, and/or CBN; or
d. a method of reducing food effects in administration of a cannabinoid, e.g., CBD, CBG, and/or CBN;
comprising administering the cannabinoid, e.g., CBD, CBG, and/or CBN, in the form of a self-emulsifying composition, e.g., according to any of Composition 1, et seq.

The self-emulsifying composition of the present invention contains an active pharmaceutical ingredient (API) containing cannabidiol (CBD), e.g., a mixture of CBD, CBG and CBN. In certain embodiments, the active pharmaceutical ingredient (API) is free or substantially free of delta-9-tetrahydrocannabinol (THC), e.g. contains less than 0.03%, less than 0.01%, less than 0.001%, less than 0.0001%, or less than 0.00001% THC by weight of the API. In some embodiments, the API is a CBD-rich hemp extract. As used herein, the term “hemp” refers to cannabis that contains 0.3% or less THC by dry weight. The CBD-rich hemp extract may be prepared by any method known in the art. For example, cannabinoids may be extracted from dried hemp leaves of the three species Cannabis sativa, Cannabis indica, and Cannabis ruderalis using a hydrocarbon solvent such as butane, a supercritical solvent such as carbon dioxide, or ethanol. The hemp extracts may be processed to remove THC and enrich CBD by purification steps such as column chromatography. The API may comprise other cannabidiols such as cannabigerol (CBG) and/or cannabinol (CBN). In some embodiments, the API comprises CBD, CBG and CBN, optionally wherein the CBD, CBG and CBN are present in a weight ratio of 1-10:1-10:1-10 (CBD:CBG:CBN). In other embodiments, the cannabidiol present in the API may be a synthetic cannabidiol. As used herein, the term “synthetic cannabidiol” refers to cannabidiol that is manufactured using chemical means rather than obtained from hemp plant. Cannabidiol may be manufactured by any method known in the art. In some embodiments, the API does not contain any cannabinoid other than CBD. In other embodiments, the API comprises other cannabidiols such as cannabigerol (CBG) and cannabinol (CBN). In some embodiments, the API comprises CBD, CBG and CBN, optionally wherein the CBD, CBG and CBN are present in a weight ratio of 1-10:1-10:1-10 (CBD:CBG:CBN).

The API is present in an amount of 1-15% by weight of the composition. In some embodiments, the API is present in an amount of 2-15%, 5-15%, 2-10%, 5-10%, 10-15%, 8-12%, or about 10% by weight of the composition.

The self-emulsifying composition of the present invention emulsifies spontaneously to produce fine oil-in-water emulsions upon dilution with aqueous media, e.g., water or gastrointestinal fluids. In some embodiments, the emulsions formed upon dilution with gastrointestinal fluids have an oil-phase particle-size distribution where an average oil-phase droplet diameter less than 1000 nm, less than 500 nm, less than 100 nm or less than 50 nm.

The self-emulsifying composition of the present invention comprises one or more non-polar solvents in an amount of 20-80% by weight of the composition. In some embodiments, the one or more non-polar solvents are present in an amount of 30-70%, or 35-65% by weight of the composition. The one or more non-polar solvents comprises a glyceride. The glyceride may be monoglyceride, diglyceride, triglyceride or mixtures thereof. In some embodiments, the glyceride comprises triglyceride. The glyceride may contain medium-chain fatty acid or long-chain fatty acid. As used herein, medium-chain fatty acid refers to fatty acid having from C₆ to C₁₂ carbon atoms, while long-chain fatty acid refers to fatty acid having from C₁₃ to C₂₄ carbon atoms. In some embodiments, the one or more non-polar solvents comprises a long-chain triglyceride (C₁₃-C₂₄ triglyceride). The glyceride may contain saturated or unsaturated fatty acid. For example, the non-polar solvents may be selected from sesame oil, anise oil, apricot kernel oil, apricot kernel oil PEG-6 esters, borage oil, canola oil, castor oil, castor oil polyoxyl 35, castor oil polyoxyl 40, castor oil polyoxyl 40 hydrogenated, castor oil polyoxyl 60, castor oil polyoxyl 60 hydrogenated castor oil hydrogenated, cinnamon oil, clove oil, coconut oil, coconut oil-lecithin, coconut oil fractioned, coriander oil, corn oil, corn oil PEG-6 esters (Labrafil® M 2125 CS; PEG-6-glyceryl linoleate), corn oil PEG-8 esters, cottonseed oil, cottonseed oil hydrogenated, kernel oil, kernel oil PEG-6 esters, lemon oil, mineral oil, mineral oil (light), neutral oil, nutmeg oil, olive oil, olive oil PEG-6 esters, orange oil, palm kernel oil, palm kernel oil/hydrogenated, palm kernel oil PEG-6 esters, peanut oil, peanut oil PEG-6 esters, peppermint oil, poppy seed oil, safflower oil, sunflower oil, soybean oil, soybean oil hydrogenated, soybean oil refined, triisostearin PEG-6 esters, vegetable oil, vegetable oil hydrogenated, vegetable oils glyceride hydrogenated, vegetable oil PEG esters, triolein, trilinolein, trilinolenin, glycerol esters of saturated C₈-C₁₈ fatty acids (Gelucire® 33/01), glyceryl esters of saturated C₁₂-C₁₈ fatty acids (Gelucire® 39/01 and 43/01), glyceryl behenate, glyceryl distearate, glyceryl isostearate, glyceryl laurate, glyceryl laurate/PEG-32 laurate (Gelucire® 44/14), glyceryl monooleate (Peceol®), glyceryl monolinoleate (Maisine® CC; also known as corn oil mono-, di-, and triglycerides), glyceryl mono and dicaprylocaprate (Masester E8120), glyceryl palmitate, glyceryl palmitostearate, glyceryl palmitostearate/PEG-32 (Gelucire® 50/13) palmitostearate glyceryl ricinoleate, glyceryl stearate, glyceryl stearate/PEG stearate, glyceryl stearate/PEG-32 stearate (Gelucire® 53/10), glyceryl stearate/PEG-40 stearate, glyceryl stearate/PEG-75 stearate, glyceryl stearate/PEG-100 stearate, polyglyceryl 10-oleate, polyglyceryl 3-oleate, polyglyceryl 4-oleate, polyglyceryl 10-tetralinoleate, polyoxyl 100 glyceryl stearate, and saturated polyglycolized glycerides (Gelucire® 37/02 and Gelucire® 50/02), caprylic/capric glycerides, caprylic/capric glycerides derived from coconut oil or palm seed oil (e.g. Labrafac®, Miglyol® 810, 812, Crodamol GTCC-PN, Softison® 378), propylene glycol caprylate/caprate (Labrafac® PC), propylene glycol dicaprylate/dicaprate (Miglyol® 840), medium chain (C₈/C₁₀) mono- and diglycerides (Capmul® MCM, Capmul® MCM (L)), and glycerol esters of saturated C₈-C₁₈ fatty acids (Gelucire® 33/01), and mixtures thereof. In some embodiments, the one or more non-polar solvents comprise sesame oil and glyceryl mono-, di-, and trilinoleate (Maisine® CC; corn oil mono-, di-, and triglycerides). In some embodiments, the one or more non-polar solvents comprise sesame oil, glyceryl mono-, di-, and trilinoleate (Maisine® CC; corn oil mono-, di-, and triglycerides) and glyceryl mono and dicaprylocaprate (Masester E8120). In some embodiments, the one or more non-polar solvents comprise PEG-6-glyceryl linoleate (Labrafil® M 2125 CS; corn oil PEG-6 esters).

The self-emulsifying composition of the present invention comprises one or more surfactants in an amount of 15-70% by weight of the composition. In some embodiments, the one or more surfactants are present in an amount of 20-65% by weight of the composition. A blend of surfactants can increase the stability of emulsions and create self-emulsifying drug delivery systems (SEDDS). Surfactants have a property referred to as hydrophilic-lipophilic balance (HLB), which is a measure of how hydrophilic or lipophilic a given surfactant, or surfactant blend is. The HLB of surfactants generally ranges from 0 to 20, with 0 being the most lipophilic and 20 being the most hydrophilic. Surfactants used to produce oil-in-water emulsions often fall within the range of about 8 to 16 on the HLB scale. In some embodiments, the total surfactants in the composition have an overall HLB of about 12 or higher, which assists in formation of small droplets. Non-ionic surfactants are preferred as these are less toxic than ionic surfactants. For example, the surfactants may be selected from polysorbate 20 (Tween® 20), polysorbate 80 (Tween® 80), polyethyleneglycol 660 12-hydroxystearate (Solutol® HS-15), TPGS (d-α-tocopheryl polyethylene glycol 1000 succinate), sorbitan monolaurate, sorbitan monopalmitate, sorbitan monoleate (Span® 20), sorbitan monostearate and sorbitan tristearate, polyoxyethylene-polyoxypropylene block copolymers, e.g., Poloxamer 338, Poloxamer 407, Poloxamer, 237, Poloxamer, 217, Poloxamer 124, Poloxamer 184, Poloxamer 185, almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS; PEG-6-glyceryl linoleate), corn oil PEG-8 esters (Labrafil® WL 2609 B S), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glycerol esters of saturated C₈-C₁₈ fatty acids (Gelucire® 33/01), glyceryl esters of saturated C₁₂-C₁₈ fatty acids (Gelucire® 39/01 and 43/01), glyceryl laurate/PEG-32 laurate (Gelucire® 44/14), glyceryl laurate glyceryl/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL), polyoxyl 40 hydrogenated castor oil (Cremophor® RH 40), polyoxyl 60 hydrogenated castor oil (Cremophor® RH60), PEG-8 caproate, PEG-8 caprylate, PEG-8 caprate PEG-8 laurate, PEG-8 oleate, PEG-8 stearate, PEG-9 caproate, PEG-9 caprylate, PEG-9 caprate PEG-9 laurate, PEG-9 oleate, PEG-9 stearate, PEG-10 caproate, PEG-10 caprylate, PEG-10 caprate PEG-101aurate, PEG-10 oleate, PEG-10 stearate, PEG-101aurate, PEG-12 oleate, PEG-15 oleate, PEG-20 laurate, PEG-20 oleate, caprylate/caprate diglycerides, glyceryl monooleate, glyceryl ricinoleate, glyceryl laurate, glyceryl dilaurate, glyceryl dioleate, glyceryl mono/dioleate, glyceryl caprylate/caprate, medium chain (C₈/C₁₀) mono- and diglycerides (Capmul® MCM, Capmul® MCM (L)), mono- and diacetylated monoglycerides, polyglyceryl oleate, polyglyceryl-2 dioleate, polyglyceryl-10 trioleate, polyglyceryl-10 laurate, polyglyceryl-10 oleate, and polyglyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, PEG-20 sorbitan monolaurate, PEG-20 sorbitan monopalmitate, PEG-20 sorbitan monostearate, and PEG-20 sorbitan monooleate, and mixtures thereof In some embodiments, the one or more surfactants comprise polysorbate 80. In some embodiments, the one or more surfactants comprise polysorbate 80 and polyoxyl 35 castor oil (Cremophor® EL).

In some embodiments, the self-emulsifying composition comprises, by weight: a) 8-12% API, b) 20-25% sesame oil, c) 20-25% of a mixture of glyceryl mono-, di-, and trilinoleate (or a mixture of corn oil mono-, di-, and triglycerides), c) 20-25% polysorbate 80, and d) 20-25% polyoxyl 35 castor oil.

In some embodiments, the self-emulsifying composition comprises, by weight: a) 8-12% API, b) 25-30% PEG-6-glyceryl linoleate (or corn oil PEG-6 esters), c) 29-34% polysorbate 80, and d) 29-34% polyoxyl 35 castor oil.

In some embodiments, the self-emulsifying composition comprises, by weight: a) 8-12% API, b) 34-38% sesame oil, c) 16-20% of a mixture of glyceryl mono-, di-, and trilinoleate (or a mixture of corn oil mono-, di-, and triglycerides), d) 16-20% of a mixture of glyceryl mono and dicaprylocaprate, and e) 16-20% polysorbate 80 .

In certain embodiments, the self-emulsifying composition comprises, by weight: a) about 10% API, b) about 22.5% sesame oil, c) about 22.5% of a mixture of glyceryl mono-, di-, and trilinoleate (or a mixture of corn oil mono-, di-, and triglycerides), c) about 22.5% polysorbate 80, and d) about 22.5% polyoxyl 35 castor oil.

In certain embodiments, the self-emulsifying composition comprises, by weight: a) about 10% API, b) about 27% PEG-6-glyceryl linoleate (or corn oil PEG-6 esters), c) about 31.5% polysorbate 80, and d) about 31.5% polyoxyl 35 castor oil.

In certain embodiments, the self-emulsifying composition comprises, by weight: a) about 10% API, b) about 36% sesame oil, c) about 18% of a mixture of glyceryl mono-, di-, and trilinoleate (or a mixture of corn oil mono-, di-, and triglycerides), d) about 18% of a mixture of glyceryl mono and dicaprylocaprate, and e) about 18% polysorbate 80 .

In some embodiments, the inclusion of one or more non-polar antioxidants may be beneficial to slow or halt the degradation of the non-polar active ingredient(s). These oil-soluble antioxidants are ideally selected from mixed tocopherols, quercetin, curcumin or other suitable oil-soluble antioxidants, with the ability to slow or halt the degradation of the non-polar active ingredient(s). In some embodiments, the addition of anti-bacterial and/or anti-fungal agents may be beneficial. These anti-bacterial and/or anti-fungal agents are ideally non-polar, natural anti-bacterial and/or anti-fungal agents selected from among thymol, carvacrol, lauric acid, or other naturally occurring anti-bacterial and/or anti-fungal agents.

The disclosure further provides a pharmaceutical composition comprising a self-emulsifying composition as described herein, e.g., any of Compositions 1 et seq. The pharmaceutical composition is an oral dosage form. The oral dosage form may be a capsule e.g., a soft gelatin capsule, a hard gelatin capsule, or a hydroxypropylmethyl cellulose (HPMC) capsule, a powder or a tablet. In some embodiments, the self-emulsifying composition is liquid and is filled into a capsule.

In another embodiment, the disclosure provides a method of treating a condition selected from pain, anxiety, nausea, dysphoria, insomnia, neuroinflammation, spasms, epilepsy, inflammation, Alzheimer's Disease, Amyotrophic Lateral Sclerosis (ALS), chronic pain, Diabetes Mellitus, dystonia, epilepsy, fibromyalgia, gastrointestinal disorders, gliomas/cancer, Hepatitis C, Human Immunodeficiency Virus (HIV), Huntington Disease, Hypertension, Incontinence, Methicillin-resistant Staphyloccus aureus (MRSA), Multiple Sclerosis, osteoporosis, pruritus, rheumatoid arthritis, sleep apnea, Parkinson's disease, chronic inflammation, chronic pain, cancer, nausea, vomiting, obesity, epilepsy, glaucoma, asthma, mood disorders, and Tourette Syndrome, comprising administering an effective amount of a pharmaceutical composition comprising a self-emulsifying composition as described herein, e.g., any of Compositions 1 et seq. to a patient in need thereof.

For example, the disclosure provides a method of treating a condition selected from pain, anxiety, nausea, insomnia, and dysphoria comprising administering an effective amount of a pharmaceutical composition comprising a self-emulsifying composition as described herein, e.g., any of Compositions 1 et seq. to a patient in need thereof.

For example, the disclosure provides a method of treating a condition selected from pain, anxiety, nausea, insomnia, and dysphoria comprising administering an effective amount of a pharmaceutical composition comprising a self-emulsifying composition as described herein, e.g., any of Compositions 1 et seq. to a patient in need thereof, wherein the API is administered in a daily dose of 20-100 mg of cannabinoid, e.g., wherein the API is administered in a daily dose comprising 10-50 mg CBD; or 10-50 mg CBD and 1-5 mg CBG; or 10-50 mg CBD and 1-5 mg CBN; or 10-50 mg CBD, 1 — 5 mg CBG, and 1-5 mg CBN.

Many phytocannabinoids act as reuptake inhibitors which allow endogenous cannabinoids to persist longer. While THC binds directly to the cannabinoid receptors, primarily CB1, the minor cannabinoids, including CBD, seem to be important for reuptake inhibition. A WHO assessment shows that CBD has low affinity to CB1 and CB2 but promotes activity of endocannabinoids. Paracetamol is also associated with endocannabinoid effects (FAAH receptors), although the details are not well-understood. There is also interaction between the cannabinoid system and the opiate system, so these cannabinoids may potentiate action of opioids, allowing for lower dose and reduced risk of addition.

The disclosure thus provides a method of relieving pain in a patient in need thereof, comprising administering an effective amount of a pharmaceutical composition comprising a self-emulsifying composition as described herein, e.g., any of Compositions 1 et seq. in combination with opioid, wherein the coadministration of cannabidiol lowers the dose of opioid required to relieve pain.

Unless stated otherwise, all percentages of composition components given in this specification are by weight based on a total composition or formulation weight of 100%.

The compositions and formulations as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

### EXAMPLES

### Example 1: Stabilit), and Dispersion of Test Formulations

Three different formulations (Formulations 1-3) for filling into soft-gel capsules are prepared as indicated in Table 1.

TABLE 1

| | Formulation 1 | Formulation 2 | Formulation 3 |
|---|---|---|---|
| Component (wt. %) | (IN07) | (IN09) | (IN17) |
| CBD¹ (wt. %) | 10 | 10 | 10 |
| Sesame oil (wt. %) | 22.5 | | 36 |
| Maisine CC² (wt. %) | 22.5 | | 18 |
| Labrafil M2125 | | 27 | |
| CS oil³ (wt. %) | | | |
| Masester E8120⁴ (wt. %) | | | 18 |
| Tween 80⁵ (wt. %) | 22.5 | 31.5 | 18 |
| Cremophor EL⁶ (wt. %) | 22.5 | 31.5 | |

¹Cannabidiol
²Mono-, di- and triglycerides of mainly linoleic (C_{18:2}) and oleic (C_{18:1}) acids, the diester fraction being predominant (Gattefossé)
³Mono-, di- and triglycerides and PEG-6 (MW 300) mono- and diesters of linoleic (C_{18:2}) acid (Gattefossé)
⁴Glyceryl mono- and dicaprylocaprate
⁵Polysorbate 80 (Sigma-Aldrich)
⁶Polyoxyl-35 castor oil, i.e., polyethoxylated castor oil made by reacting castor oil with ethylene oxide in a molar ratio of 1:35 (BASF)

Stability to moisture is measured by mixing with water. At 7 and 14 days at 3% water, phase separation is observed in Formulations 1 and 2, and some precipitation is observed in Formulation 2. At 7 and 14 days at 5% water, phase separation is observed in all three formulations, and some precipitation is observed in Formulation 2. Stability to temperature variations is measured by cycling the formulations through high temperature (40° C.) and low temperature (−20° C.) for 7 days. No phase separation or precipitation is observed in any of the formulations in response to exposure to high or low temperatures.

Dispersion of the formulations in simulated gastric and intestinal fluids is evaluated over a 12 hour period. In Fasted State Simulated Gastric Fluid (FaSSGF, pH 1.2), there is no dispersion of unformulated CBD control, rapid and complete dispersion of Formulations 1 and 2, and some dispersion (30-40%) of Formulation 3. In Fasted State Simulated Intestinal Fluid (FaSSIF, pH 6.5), there is some dispersion of CBD control (about 20%), about 60% dispersion of Formulation 3, and rapid and complete dispersion of Formulations 1 and 2. In Fed State Simulated Intestinal Fluid (FeSSIF, pH 5.5), there is some dispersion of CBD control (about 20% after 12 hours), about 80% dispersion of Formulation 3, and complete dispersion of Formulations 1 and 2, with Formulation 2 exhibiting somewhat more rapid dispersion than Formulation 1.

Based on the completion of fill evaluation of CBD control and the three formulations, Formulations 1 and 2 show a preferable dispersion pattern in the biorelevant media (FaSSIF, FeSSIF, and FaSSGF) when compared to Formulation 3. The CBD control shows poor dispersion in all of the biorelevant media. All three fill formulations are compatible with glycerin and with a sorbitol-glycerin blend, showing that the fill will not migrate into the shell (glycerin and/or sorbitol are often used as plasticizers in soft gelatin capsules).

Accordingly, these formulations, particularly Formulations 1 and 2, when administered in soft gel capsules, are expected to form self-emulsified dispersions in the stomach and intestine.

### Example 2: Bioavailability of Test Formulations

Differences in absorption from the GI tract of rats given several different preparations of cannabidiol (CBD) are evaluated. It is believed that, with a suitable formulation, the lipophilic CBD can be preferentially directed (channeled) towards lymphatic absorption versus portal vein absorption, thus avoiding metabolism in the liver and resulting in higher systemic blood levels.

Male adult rats with cannulated intestinal lymph duct and portal vein have their samples collected after administering equidose quantities of 4 solubilized or emulsified preparations. In order to get systemic pharmacokinetics, another set of rats with only jugular vein cannula is studied under the same conditions. In both groups of animals, a gastric infusion tube is introduced into the stomach through a fundal incision and the incision is sealed off by a purse string suture.

For Experiment 1, five days before an experiment is planned, the animal undergoes surgery for the installation of various cannulas. Under isoflurane anesthesia, the portal vein is cannulated. The animal is then allowed to recover for 3 days prior to the intestinal lymph duct cannulation. A stomach infusion tube is also installed. After overnight recovery, the lymph catheter collects chylomicrons produced and secreted by the small intestinal epithelial cells (enterocytes). The portal vein allows blood sampling when lymph samples are collected. This allows a comparison of lymphatic versus portal transport of CBD when infused with different vehicles. Rats with satisfactory catheters are used for each test article. Each rat receives an equal dose of the test article to which they have been assigned as a slow gavage five minutes. Lymph samples are sampled for 1 h before gavage and then hourly for 6 h. In certain experiments, 30 min. lymph samples are taken to present a better picture of the lymphatic transport. Other experiments to go more than 6 h to 8 h. At each hour time point of lymph collection 0.2 ml of blood is taken from the portal vein to isolate the plasma for analyses. The lymphatic flow usually varies between 2-4 ml per hour.

In Experiment 2, animals are fasted overnight prior to surgery. In the morning, under isofluorane anesthesia, the jugular vein is cannulated and the stomach is intubated as described above. Similar mixture as those tested in Experiment 1 is administered into the stomach and blood samples is collected as portal vein samples described above and the plasma is analyzed for CBD content. Jugular vein blood (0.2 ml) is taken from the rats and analyzed for CBD content.

Blood collected from the portal and jugular catheters is quickly spun and plasma is collected, frozen, and stored at −80 C. Intestinal lymph samples are frozen and stored at −80 C. Some of this lymph is fractionated into chylomicron and other fractions for CBD assay using LC-MS.

The concentrations of CBD for each animal at each interval is plotted for each animal and summarized across all animals in a cohort for mean, SD, and SEM. The portal vein vs. thoracic duct concentrations within each group are compared with a T test. The portal vein, thoracic duct and systemic values among the four groups are compared with ANOVA testing.

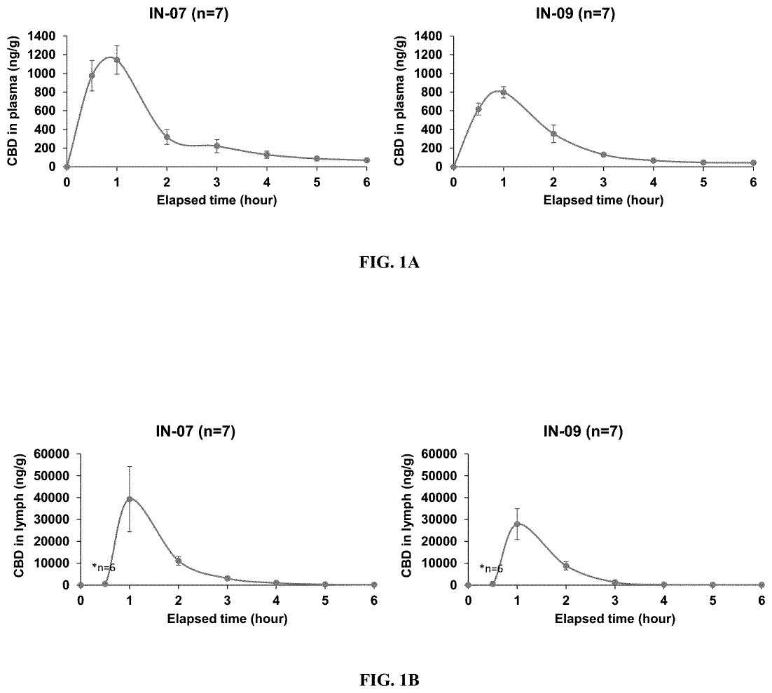

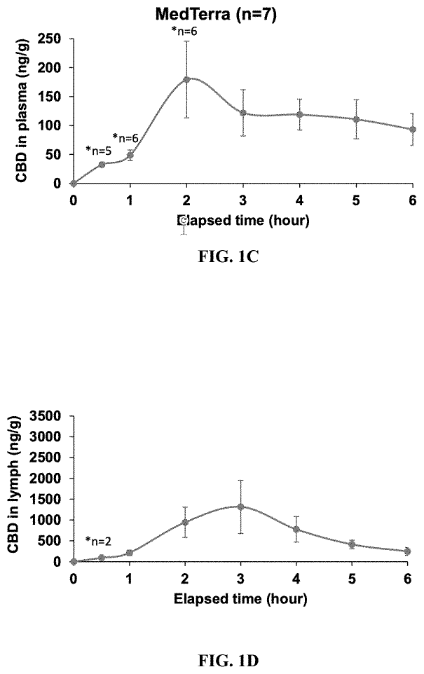

FIG. 1A depicts levels of CBD in portal vein plasma for Formulation 1 (IN-07) and Formulation 2 (IN-09). The Cmax for IN-07 was 1144.19 ng/g at 1 hr. The Cmax in IN-09 was 797.32 ng/g, also at 1 hr. The results are set forth in the table below.

| | Time | | |
|---|---|---|---|
| | point | AVE | SEM |
| Samples | (hour) | (ng/g) | (ng/g) |
| IN07_Pre-Infustion | 0 | 0 | 0 |
| IN07_0.5 hour | 0.5 | 974.96 | 163.28 |
| IN07_1 hour | 1 | 1144.19 | 154.23 |
| IN07_2 hour | 2 | 319.42 | 80.06 |
| IN07_3 hour | 3 | 221.39 | 70.37 |
| IN07_4 hour | 4 | 129.60 | 38.33 |
| IN07_5 hour | 5 | 87.38 | 24.23 |
| IN07_6 hour | 6 | 69.71 | 15.15 |
| IN09_Pre-infustion | 0 | 0 | 0 |
| IN09_0.5 hour | 0.5 | 651.72 | 64.55 |
| IN09_1 hour | 1 | 797.32 | 59.83 |
| IN09_2 hour | 2 | 353.31 | 94.11 |
| IN09_3 hour | 3 | 130.48 | 17.54 |
| IN09_4 hour | 4 | 67.91 | 8.09 |
| IN09 5 hour | 5 | 46.11 | 3.95 |
| IN09_6 hour | 6 | 43.18 | 3.01 |

FIG. 1B depicts levels of CBD in superior mesenteric lymph for Formulation 1 (IN-07) and Formulation 2 (IN-09). The Cmax for IN-07 at 1 hour was 39297.46 ng/g. The Cmax for IN-09 at 1 hour was 27876.33 ng/g. The results are set forth in the table below.

| | Time | | |
|---|---|---|---|
| | point | AVE | SEM |
| Samples | (hour) | (ng/g) | (ng/g) |
| IN07_Pre-Infustion | 0 | 0 | 0 |
| IN07_0.5 hour | 0.5 | 489.52 | 348.27 |
| IN07_1 hour | 1 | 39297.46 | 14932.38 |
| IN07_2 hour | 2 | 11092.77 | 1984.30 |
| IN07_3 hour | 3 | 3038.24 | 876.30 |
| IN07_4 hour | 4 | 961.03 | 301.52 |
| IN07_5 hour | 5 | 270.63 | 73.19 |
| IN07_6 hour | 6 | 132.45 | 23.79 |
| IN09_Pre-Infustion | 0 | 0 | 0 |
| IN09_0.5 hour | 0.5 | 515.06 | 259.17 |
| IN09_1 hour | 1 | 27876.33 | 7051.83 |
| IN09_2 hour | 2 | 8812.67 | 1889.05 |
| IN09_3 hour | 3 | 1265.24 | 338.52 |
| IN09_4 hour | 4 | 192.03 | 31.75 |
| IN09_5 hour | 5 | 102.61 | 14.15 |
| IN09_6 hour | 6 | 71.97 | 8.07 |

For both of these formulations, the Cmax in lymph was much higher in lymph than in plasma. The ratio of Cmax in lymph to Cmax in plasma was 34.35 for IN-07 and 34.96 for IN-09.

A commercial formulation of CBD in a medium chain triglyceride vehicle (Medterra) in an amount containing equivalent levels of CBD is tested as a control. As depicted in FIG. 1C, this formulation provided a Cmax in portal vein plasma of 179.41 ng/g at 2 hrs. See following table:

| | Time | | |
|---|---|---|---|
| | point | AVE | SEM |
| Samples | (hour) | (ng/g) | (ng/g) |
| MedTerra_Pre-Infustion | 0 | 0 | 0 |
| MedTerra_0.5 hour | 0.5 | 32.37 | 4.12 |
| MedTerra_1 hour | 1 | 48.52 | 9.22 |
| MedTerra_2 hour | 2 | 179.41 | 66.29 |
| MedTerra_3 hour | 3 | 121.94 | 40.00 |
| MedTerra_4 hour | 4 | 118.83 | 26.62 |
| MedTerra_5 hour | 5 | 110.62 | 33.60 |
| MedTerra_6 hour | 6 | 93.27 | 27.54 |

As depicted in FIG. 1D, the Cmax in superior mesenteric lymph was 1315.24 ng/g. See following table:

| | Time | | |
|---|---|---|---|
| | point | AVE | SEM |
| Samples | (hour) | (ng/g) | (ng/g) |
| MedTerra_Pre-Infustion | 0 | 0 | 0 |
| MedTerra_0.5 hour | 0.5 | 91.33 | 31.32 |
| MedTerra_1 hour | 1 | 209.74 | 66.95 |
| MedTerra_2 hour | 2 | 942.98 | 363.92 |
| MedTerra_3 hour | 3 | 1315.24 | 639.91 |
| MedTerra_4 hour | 4 | 775.31 | 308.17 |
| MedTerra_5 hour | 5 | 413.43 | 105.15 |
| MedTerra_6 hour | 3 | 244.53 | 92.97 |

The ratio of Cmax in lymph to Cmax in plasma for the MedTerra formulation was 7.33.

The absorption for the commercial formulation vs the INO7 and INO9 test formulations was much slower, the Cmax levels both in plasma and lymph were lower, and the uptake into lymph vs. plasma was less pronounced.

As demonstrated, the formulation of CBD has effects on Cmax, Tmax and AUC. Without formulation or with suboptimal formulation, CBD absorption is slow and irregular, due to lack of gastric delivery, and lipolysis and absorption during transit through the small intestine is slow. This results in lower Cmax, slower absorption time, and unusual and/or variable PK curves. AUC is also influenced by the prolonged low-level absorption. The INO7 and INO9 formulations deliver higher Cmax, both in plasma and in lymph, than the control formulation, permitting less opportunity for individual variation and allowing delivery of therapeutically effective amounts of CBD. Moreover, it is believed that differential packaging into lymphatic chylomicrons will provide improvements in systemic PK, reduced first-pass metabolism in the liver, and delivery to specific organs and tissues.

1. A self-emulsifying composition, comprising:
(i) an active pharmaceutical ingredient (API) in an amount of 1-15% by weight of the composition;
(ii) one or more non-polar solvents in an amount of 20-80% by weight of the composition; and
(iii) one or more surfactants in an amount of 15-70% by weight of the composition,
wherein the active pharmaceutical ingredient (API) comprises cannabidiol (CBD) and wherein the one or more non-polar solvents comprise a glyceride.

2. The composition of claim 1, wherein the active pharmaceutical ingredient (API) is free or substantially free of delta-9-tetrahydrocannabinol (THC).

3. The composition of claim 1, wherein the API is a CBD-rich hemp extract.

4. The composition of claim 1, wherein the API comprises CBD, CBG and CBN, optionally wherein the CBD, CBG and CBN are present in a weight ratio of 1-10:1-10:1-10 (CBD:CBG:CBN).

5. The composition of claim 1, wherein the composition emulsifies spontaneously to produce fine oil-in-water emulsions upon dilution with aqueous media.

6. The composition of claim 1, wherein the emulsion formed upon dilution with gastrointestinal fluids has an oil-phase particle-size distribution where an average oil-phase droplet diameter less than 1000 nm.

7. The composition of claim 1, wherein the glyceride is monoglyceride, diglyceride, triglyceride or mixtures thereof.

8. The composition of any preceding claim 1, wherein the one or more non-polar solvents comprises triglyceride.

9. The composition of claim 1, wherein the one or more non-polar solvents comprises a triglyceride having C₁₃ -C₂₄ fatty acids, e.g. C₁₆-C₂₂ fatty acids, e.g., C₁₆ and/or C₁₈ fatty acids.

10. The composition of any preceding claim, wherein the one or more surfactants comprise polysorbate 80, polyoxyl 35 castor oil or a combination thereof.

11. The composition of claim 1, wherein the self-emulsifying composition comprises, by weight: a) 8-12% API; b) 20-25% sesame oil; c) 20-25% of a mixture of mono-, di-, and triglycerides of corn oil fatty acids (e.g., a mixture of mono-, di- and triglycerides of mainly linoleic (C_{18:2}) and oleic (C_{18:1}) acids, the diester fraction being predominant); c) 20-25% polysorbate 80; and d) 20-25% polyoxyl 35 castor oil.

12. The composition of claim 1, wherein the self-emulsifying composition comprises, by weight: a) 8-12% API, b) 25-30% mono-, di- and triglycerides and PEG-6 (MW 300) mono- and diesters of linoleic (C_{18:2}) acid; c) 29-34% polysorbate 80; and d) 29-34% polyoxyl 35 castor oil.

13. The composition of claim 11, wherein the self-emulsifying composition comprises, by weight: a) about 10% CBD, b) about 22.5% sesame oil, c) about 22.5% of a mixture of mono-, di- and triglycerides of mainly linoleic (C_{18:2}) and oleic (C_{18:1}) acids, c) about 22.5% polysorbate 80, and d) about 22.5% polyoxyl 35 castor oil.

14. The composition of claim 12, wherein the self-emulsifying composition comprises, by weight: a) about 10% CBD; b) about 27% mono-, di- and triglycerides and PEG-6 (MW 300) mono- and diesters of linoleic (C_{18:2}) acid; c) about 31.5% polysorbate 80, and d) about 31.5% polyoxyl 35 castor oil.

15. The composition of claim 1, wherein, upon oral administration, at least 40%, of the API absorbed is absorbed via the lymphatic system rather than the portal vein absorption.

16. A pharmaceutical composition comprising a self-emulsifying composition according to any preceding claim, wherein the pharmaceutical composition is an oral dosage form.

17. A method of treating a condition selected from pain, anxiety, nausea, insomnia, and dysphoria comprising administering an effective amount of a pharmaceutical composition of claim 16 to a patient in need thereof.

18. A method of relieving pain in a patient in need thereof, comprising administering an effective amount of a pharmaceutical composition of claim 16 in combination with an opioid, wherein the coadministration of cannabidiol lowers the dose of opioid required to relieve pain.
